(19) United States
(12) Patent Application Publication
(10) Pub. No.: US 2025/0351859 A1
(43) Pub. Date: Nov. 20, 2025

(54) METHOD FOR MANUFACTURING PERSONALIZED NUTRITIONAL COMPOSITION WITH PRECISELY CONTROLLED NUTRIENT CONTENT VIA EMBEDDED 3D PRINTING

(71) Applicant: TOP TABLE INC, Seoul (KR)

(72) Inventor: Hyun Ju YOO, Seoul (KR)

(73) Assignee: TOP TABLE INC, Seoul (KR)

(21) Appl. No.: 19/281,268

(22) Filed: Jul. 25, 2025

Related U.S. Application Data
(63) Continuation of application No. PCT/KR2024/001224, filed on Jan. 25, 2024.

(30) Foreign Application Priority Data
Jan. 31, 2023 (KR) 10-2023-0012880

(51) Int. Cl. A23L 33/00; A23P 10/25; B33Y 10/00; B33Y 70/00; B33Y 80/00

(52) U.S. Cl. CPC A23L 33/30; A23P 10/25; B33Y 10/00; B33Y 70/00; B33Y 80/00

(57) ABSTRACT

The present disclosure relates to a method for preparing a personalized nutritional supplement composition having precisely controlled nutrient content through embedded 3D printing, which can derive nutritional requirement information based on biometric information and dietary activity information of a user and provide a personalized nutritional supplement composition including droplets containing nutrients required by the user.

### CROSS-REFERENCE TO RELATED APPLICATIONS

This application is a continuation of International Application No. PCT/KR2024/001224 filed on Jan. 25, 2024, which claims priority to Korean Patent Application No. 10-2023-0012880 filed on Jan. 31, 2023, the entire contents of which are herein incorporated by reference.

### TECHNICAL FIELD

The present disclosure relates to a method for preparing a personalized nutritional supplement composition having precisely controlled nutrient content through embedded 3D printing.

### BACKGROUND ART

The personalized nutrition healthcare market has recently seen development across multiple sectors including wearable tech, diagnostics, consumer goods, medical devices, and healthcare services, with rapid expansion driven by the emergence of functional health foods offering targeted benefits like weight management or tailored vitamin supplementation.

However, current healthcare manufacturing systems remain largely limited to assembling pre-existing components using traditional formulation approaches. These systems rely heavily on imprecise manual processes, resulting in products that vary only in basic attributes like appearance and nutrient profiles based on raw material differences, without reaching the stage of producing truly customized products for actual consumers.

In the context of nutritional supplements, ‘formulation’ refers to ‘forms manufactured and processed for ease of consumption’, literally meaning forms that are convenient to consume. The Korean government has recently embraced formulation diversification to meet consumer demands, including the decision to permit ‘gummy’ formats for vitamin and mineral pharmaceuticals under the ‘6th New Industry Field Regulatory Innovation Plan’.

These formulations represent far more than mere shape-forming technology, requiring careful consideration of the physicochemical properties of functional ingredients, including stability, solubility, and interactions with additives, while also accounting for safety from environmental factors such as light, moisture, and temperature during storage as well as consumer preference factors.

Consequently, there exists a clear need for research into novel formulation approaches that enable precise nutrient control while incorporating enhanced functional benefits for nutritional supplements.

The inventors of the present disclosure have completed this invention after extensive research and iterative development aimed at addressing the aforementioned challenges.

### SUMMARY

### Technical Problem

To address the aforementioned challenges, the present disclosure aims to provide a method for preparing a personalized nutritional supplement composition with precisely controlled nutrient content through embedded 3D printing technology. This approach involves forming droplets containing bioactive compounds and vitamins, then controlling their physical properties to enable embedded formation within a nutritional supplement composition base, thereby enabling manufacture of nutritional supplement compositions in novel formulation formats.

The technical problems to be solved by the present disclosure are not limited to the aforementioned technical problem, and other technical problems not mentioned herein will be clearly understood by those skilled in the art from the following description.

### Solution to Problem

One aspect of the present disclosure for addressing the aforementioned challenges includes: (a) deriving nutritional requirement information based on biometric information and dietary activity information of a user; (b) setting a minimum controllable unit for each nutrient to be introduced into a nutritional supplement composition in the form of a droplet, based on the derived nutritional requirement information; (c) calculating, with respect to the nutritional supplement composition, a weight percentage of each type of droplet containing a nutrient for which the minimum controllable unit has been set; (d) assigning a unique color to each nutrient and incorporating the assigned unique color; and (e) forming droplets within a liquid or gel-based nutritional supplement composition base through embedded 3D printing.

### Advantageous Effects of Invention

The present disclosure provides the effect of delivering a novel formulation nutritional supplement composition in which droplets are fixed within a nutritional supplement composition base by controlling the viscosity and gelation properties of the nutritional supplement composition base.

The present disclosure provides the effect of enabling fine-scale control of nutrient content through configuration of droplet concentration, volume, position, and number.

The present disclosure provides the effect of delivering a personalized nutritional supplement composition through an embedded 3D printing system that is linked with user nutritional requirement data.

The present disclosure provides the effect of enabling users to visually identify required nutrients by setting droplet colors according to the nutrients contained in the droplets included in the nutritional supplement composition.

Furthermore, it is noted that even effects not explicitly mentioned herein, including effects described in the following specification that are expected from the technical features of the present disclosure and their potential effects, are treated as disclosed in the specification of the present disclosure.

### DESCRIPTION OF DRAWINGS

FIG. 1 is a reference diagram showing nutritional requirement information derived for different users.

FIG. 2 is a reference diagram showing degree of nutrient diffusion relative to hydrophobicity of droplets according to an embodiment of present disclosure.

FIG. 3 is a reference diagram showing formability and sedimentation/floating characteristics of droplets according to an embodiment of present disclosure.

FIG. 4 is a reference diagram showing changes in droplets according to nozzle movement speed and flow rate of embedded 3D printing device performing continuous dispensing.

### DETAILED DESCRIPTION

One aspect of the present disclosure for addressing the aforementioned challenges includes: (a) deriving nutritional requirement information based on biometric information and dietary activity information of a user; (b) setting a minimum controllable unit for each nutrient to be introduced into a nutritional supplement composition in the form of a droplet, based on the derived nutritional requirement information; (c) calculating, with respect to the nutritional supplement composition, a weight percentage of each type of droplet containing a nutrient for which the minimum controllable unit has been set; (d) assigning a unique color to each nutrient and incorporating the assigned unique color; and (e) forming droplets within a liquid or gel-based nutritional supplement composition base through embedded 3D printing.

According to a preferred embodiment of the present disclosure, the step (e) may include forming droplets by injecting a nutrient solution including a hydrophobic substance that forms a spherical shape by self-cohesion and surface tension when embedded in the nutritional supplement composition base.

According to a preferred embodiment of the present disclosure, the step (e) may include: setting a size and a formation position of the droplets within the nutritional supplement composition base through nozzle control of an embedded 3D printing device; and setting a quantity to be incorporated into the nutritional supplement composition for each type of droplet including a different nutrient.

According to a preferred embodiment of the present disclosure, the step (e) may include setting a size and volume of the droplets through control of a movement speed and a flow rate of the nozzle.

According to a preferred embodiment of the present disclosure, the step (e) may include: setting a density of the nutritional supplement composition base to be similar to that of the droplets to fix the droplets formed within the nutritional supplement composition base at specific positions; and setting a viscosity of the nutritional supplement composition base to a certain level or above in order to fix the droplets formed within the nutritional supplement composition base at specific positions.

According to a preferred embodiment of the present disclosure, the step (d) may include forming the droplets using a nutrient solution incorporated with the unique color, thereby enabling a user to visually identify a required nutrient.

### MODE FOR INVENTION

In describing the present disclosure, detailed descriptions of related known functions that are obvious to those skilled in the art and that may unnecessarily obscure the gist of the present disclosure are omitted.

The method for preparing a personalized nutritional supplement composition with precisely controlled nutrient content through embedded 3D printing according to the present disclosure enables provision of personalized nutritional supplement compositions to each user based on nutritional information required by the user.

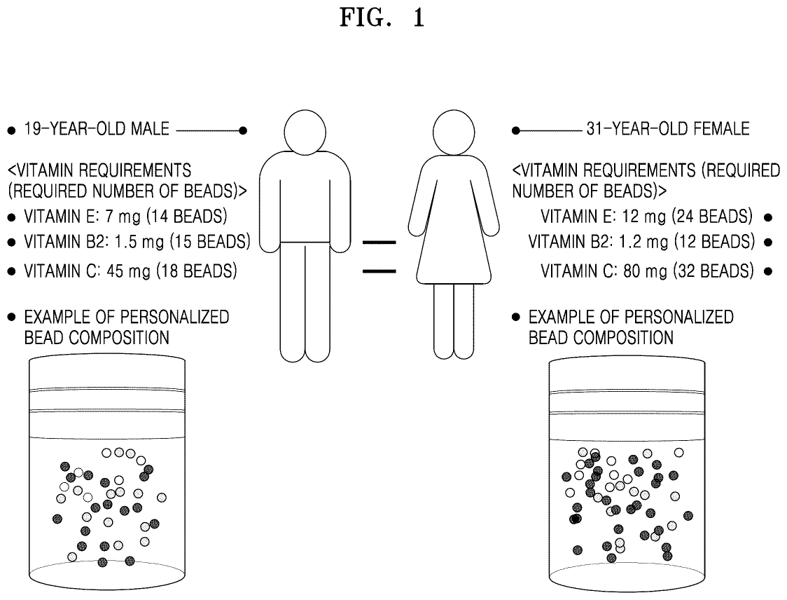

In this regard, FIG. 1 shows nutritional requirement information derived for different users and the corresponding forms of personalized nutritional supplement compositions.

Referring to FIG. 1, the present disclosure can form droplets containing at least one required nutrient based on nutritional requirement information derived for different users.

Furthermore, through precise control in 0.1 mg increments, the size and volume of droplets may be determined, and the number of droplets according to the user's vitamin requirements may be calculated to enable manufacture of personalized nutritional supplement compositions.

The method for preparing a personalized nutritional supplement composition having precisely controlled nutrient content through embedded 3D printing according to the present disclosure may be performed by a nutritional supplement composition manufacturing apparatus including an embedded 3D printing device, and largely comprises the following steps (a) to (e).
(a) deriving nutritional requirement information based on biometric information and dietary activity information of a user;
(b) setting a minimum controllable unit for each nutrient to be introduced into the nutritional supplement composition in the form of a droplet, based on the derived nutritional requirement information;
(c) calculating, with respect to the nutritional supplement composition, a weight percentage of each type of droplet containing a nutrient for which the minimum controllable unit has been set;
(d) assigning a unique color to each nutrient and incorporating the assigned unique color; and
(e) forming droplets within a liquid or gel-based nutritional supplement composition base through embedded 3D printing.

According to an embodiment of the present disclosure, the step (d) may include forming the droplets using a nutrient solution incorporated with the unique color, thereby enabling a user to visually identify a required nutrient.

According to an embodiment of the present disclosure, the step (e) may include forming droplets by injecting a nutrient solution comprising a hydrophobic substance that forms a spherical shape by self-cohesion and surface tension when embedded in the nutritional supplement composition base.

Herein, the droplets according to the present disclosure are materials that contain nutrients while simultaneously preventing leakage into the nutritional supplement composition base, and are not particularly limited to specific substances.

For example, non-polar molecules including vegetable oil, essential oil, and water-in-oil emulsions, or biopolymers capable of film formation under the influence of metal ions, such as alginic acid, pectin, and carrageenan, may all be included in the droplets.

Furthermore, in each embodiment according to the present disclosure, to determine the volume, shape, independence, and organoleptic properties of the droplets, droplets were formed by embedded 3D printing of a nutrient solution containing 0.1 to 20 wt % of medium-chain triglyceride (MCT) oil and functional substances into a gel-transition nutritional supplement composition base containing hydroxypropyl methylcellulose (HPMC) at a 0.5 to 5% ratio and gelatin at a 10% ratio.

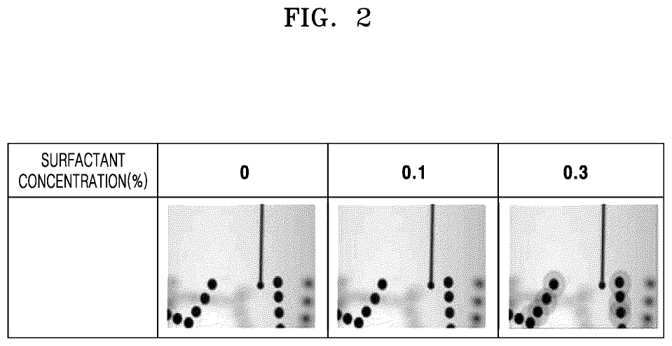

FIG. 2 is a reference diagram showing the degree of nutrient diffusion relative to the hydrophobicity of droplets according to an embodiment of the present disclosure.

Referring to FIG. 2, the degree of diffusion of vitamin B12 captured in droplets into the nutritional supplement composition base was measured according to the hydrophobicity that changes with the level of surfactant incorporation in the droplets and according to the hydrophobic changes.

The measurement results show that as the degree of hydrophilic/hydrophobic difference between n the nutritional supplement composition base and droplets decreases with surfactant incorporation, the vitamins captured in the droplets diffuse into the nutritional supplement composition base.

At surfactant concentrations of 0.1% or less, it was observed that the captured vitamins did not diffuse into the nutritional supplement composition base and maintained a well-aggregated form (droplets), but at surfactant concentrations of 0.3% or more, diffusion proceeded and aggregation was inhibited.

According to this embodiment, it can be seen that droplet formation is affected not only by the specific gravity difference between the droplets and the nutritional supplement composition base and the viscosity of the nutritional supplement composition base, but also by the divergence rate of hydrophilic/hydrophobic properties.

According to the above results, it can be seen that in the case of nutritional supplement compositions where multiple nutrients are individually captured in multiple droplets, bioactive functions may be impaired due to inter-material interactions and visual effects may be lost.

According to an embodiment of the present disclosure, the step (e) may include: setting a density of the nutritional supplement composition base to be similar to that of the droplets to fix the droplets formed within the nutritional supplement composition base at specific positions; and setting a viscosity of the nutritional supplement composition base to a certain level or above in order to fix the droplets formed within the nutritional supplement composition base at specific positions.

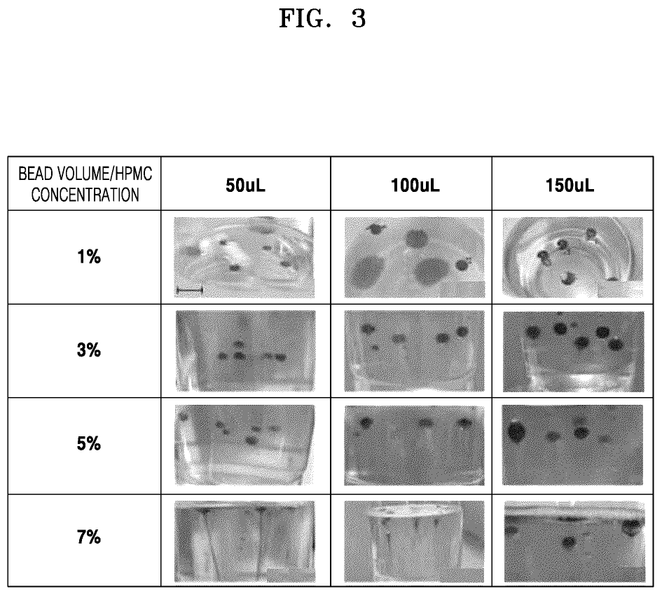

In this regard, FIG. 3 is a reference diagram showing the formability and sedimentation/floating characteristics of droplets according to an embodiment of the present disclosure.

Referring to FIG. 3, the formability and sedimentation/floating characteristics of droplets were measured according to the viscosity that changes with the amount of HPMC incorporation in the nutritional supplement composition base and the volume of the droplets.

First, the diameter of the droplets increases as the input amount of MCT-based nutrient solution inserted into the nutritional supplement composition base increases. In this embodiment, precise control in 0.5 mm increments was performed.

This means that by adjusting the diameter of the droplets, the content of nutrients contained in each droplet and the visual effects can be freely modified.

Furthermore, as a result of testing the floating characteristics of droplets according to HPMC concentration, droplets formed at HPMC concentrations below 3% floated away from their original positions where the nutrient solution was injected, and phase separation due to aggregation with adjacent droplets was observed during the floating process.

Meanwhile, droplets with volumes of 150 μl or less formed at HPMC concentrations of 3% or more were well fixed at designated positions without sedimentation or floating. Accordingly, it was confirmed that HPMC concentrations of 3% or more can provide sufficient internal resistance (viscosity) to suppress floating of droplets with volumes of 150 μl or less. However, droplets with volumes of 150 μl or less formed at HPMC concentrations of 7% or more exhibited phenomena where they floated away from their original injected positions or failed to maintain their shape.

According to the above results, it can be seen that to suppress phase separation between droplets and the nutritional supplement composition base, it is preferable to utilize a liquid or gel-phase nutritional supplement composition base that minimizes density differences or has sufficient resistance (viscosity) to overcome density differences.

Furthermore, in a nutritional supplement composition base containing HPMC at concentrations of 3% or more and less than 7%, each droplet entity can be positioned independently at designated locations without aggregating with each other, so it can be seen that it is preferable to form droplets with volumes of 150 μl or less in a nutritional supplement composition base incorporating HPMC at concentrations of 3% or more and less than 7%.

According to an embodiment of the present disclosure, the step (e) may include setting the size of droplets and their formation positions within the nutritional supplement composition base through nozzle control of the embedded 3D printing device; and setting the quantity to be incorporated into the nutritional supplement composition for each type of droplet containing different nutrients.

Specifically, it may include setting the size and volume of droplets through control of the nozzle movement speed and flow rate.

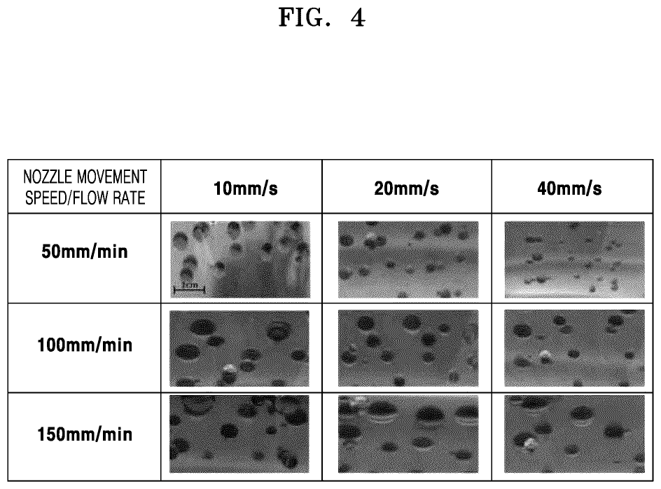

FIG. 4 is a reference diagram showing changes in droplets according to nozzle movement speed and flow rate of an embedded 3D printing device performing continuous dispensing.

Referring to FIG. 4, the effects of flow rate and nozzle movement speed on the volume and resolution of droplets embedded within a liquid or gel-phase nutritional supplement composition base through embedded 3D printing performing continuous injection were measured.

As shown in FIG. 4, it can be confirmed that hydrophobic substances continuously injected within a liquid or gel-phase nutritional supplement composition base form droplets due to cohesive forces and surface tension.

This means that embedded 3D printing performed in conjunction with continuous injection can be applied to reduce the complexity of software control in droplet positioning and nutrient content control and to shorten the delay time occurring during the injection start and stop processes of the device.

According to this embodiment, it can be confirmed that the size of droplets formed decreases as the nozzle movement speed of the embedded 3D printing device increases at the same flow rate.

Furthermore, it was observed that the volume of droplets increases as the flow rate increases at the same nozzle movement speed.

This means that the volume and position of droplets can be modified in real-time according to requirements through changes in flow rate and nozzle movement speed in embedded 3D printing.

It is apparent to those skilled in the art that the present disclosure may be embodied in other specific forms without departing from the characteristics of the present disclosure. Therefore, the above detailed description should not be interpreted as restrictive in all respects but should be considered as exemplary. The scope of the present disclosure should be determined by reasonable interpretation of the appended claims, and all modifications within the equivalent scope of the present disclosure are included in the scope of the present disclosure.

1. A method for preparing a personalized nutritional supplement composition having precisely controlled nutrient content through embedded 3D printing, the method comprising:
(a) deriving nutritional requirement information based on biometric information and dietary activity information of a user;
(b) setting a minimum controllable unit for each nutrient to be introduced into a nutritional supplement composition in the form of a droplet, based on the derived nutritional requirement information;
(c) calculating, with respect to the nutritional supplement composition, a weight percentage of each type of droplet containing a nutrient for which the minimum controllable unit has been set;
(d) assigning a unique color to each nutrient and incorporating the assigned unique color; and
(e) forming droplets within a liquid or gel-based nutritional supplement composition base through embedded 3D printing.

2. The method of claim 1,
wherein the step (e) includes forming droplets by injecting a nutrient solution comprising a hydrophobic substance that forms a spherical shape by self-cohesion and surface tension when embedded in the nutritional supplement composition base.

3. The method of claim 1,
wherein the step (e) includes: setting a size and a formation position of the droplets within the nutritional supplement composition base through nozzle control of an embedded 3D printing device; and setting a quantity to be incorporated into the nutritional supplement composition for each type of droplet including a different nutrient.

4. The method of claim 3,
wherein the step (e) includes setting a size and volume of the droplets through control of a movement speed and a flow rate of a nozzle.

5. The method of claim 1,
wherein the step (e) includes:
setting a density of the nutritional supplement composition base to be similar to that of the droplets to fix the droplets at specific positions within the nutritional supplement composition base; and
setting a viscosity of the nutritional supplement composition base to a certain level or above in order to fix the droplets at specific positions within the nutritional supplement composition base.

6. The method of claim 1,
wherein the step (d) includes forming the droplets using a nutrient solution incorporated with a unique color so that a nutrient required by the user is visually identifiable.
